(19) United States
(12) Patent Application Publication
(10) Pub. No.: US 2024/0180797 A1
(43) Pub. Date: Jun. 6, 2024

(54) COMPOSITIONS AND METHODS FOR TREATING KERATIN SUBSTRATES

(71) Applicant: L'Oreal, Paris (FR)

(72) Inventors: Aakash PAREKH, Edison, NJ (US); Ronak RUGHANI, Edison, NJ (US)

(21) Appl. No.: 18/518,827

(22) Filed: Nov. 24, 2023

Related U.S. Application Data
(60) Provisional application No. 63/427,549, filed on Nov. 23, 2022.

(30) Foreign Application Priority Data
Feb. 3, 2023 (FR) 2301019

(51) Int. Cl. A61K 8/19; A61K 8/34; A61Q 5/12

(52) U.S. Cl. CPC A61K 8/19; A61K 8/345; A61Q 5/12

(57) ABSTRACT

The disclosure relates to compositions and methods for treating keratinous substrates, such as skin and/or hair, where the compositions comprise (a) at least one alkaline carbonate, (b) at least one polyol, (c) water and/or at least one non-aqueous solvent, and (d) optionally at least one pH adjuster. The at least one carbonate and the at least one polyol are present in the composition in the form of an associated network.

### CROSS REFERENCE TO RELATED APPLICATIONS

This application claims priority to U.S. Provisional Application No. 63/427,549, filed Nov. 23, 2022, and French Application No. FR 2301019, filed Feb. 3, 2023, both of which are incorporated herein.

### TECHNICAL FIELD

The present disclosure relates to compositions and methods for treating keratin substrates, such as skin or hair.

### BACKGROUND

Consumers desire to improve the appearance and/or feel of the skin and/or hair. For example, people who have dry skin or hair may wish to hydrate and/or moisturize the skin or hair in order to improve the look and feel thereof. Attributes that consumers desire include, for example, deep and lasting hydration and/or moisturizing without a greasy feeling. Additionally, improved conditioning or cosmetic benefits that last are also desired.

Current products and technologies aim to hydrate and/or moisturize skin and hair by superficially coating the surface with humectants and/or oils. Humectants linger on the surface of the skin or hair, acting as a conduit between the skin or hair and the external environment. However, while the goal is to increase moisture of the skin or hair, oftentimes, the opposite effect such as dry skin or dry, frizzy hair can occur due to non-equilibrium of the water between the skin or hair fibers and the external environment. Moreover, the superficial coating can be easily rinsed or washed off, such that long-term moisturizing and associated benefits are not achieved.

The present inventors have now surprisingly discovered a synergistic combination of ingredients that unexpectedly provides excellent moisture penetration and retention benefits to keratinous substrates such as skin and hair, which benefits are long lasting.

### SUMMARY

It has been surprisingly and unexpectedly discovered that compositions and methods according to the disclosure can increase and/or improve moisture content of the keratinous substrates, can reduce moisture loss from the keratinous substrates, and/or can provide beneficial cosmetic properties to the keratinous substrates.

In various embodiments, the disclosure relates to compositions for treating keratinous substrates, such as skin and/or hair, the compositions comprising (a) at least one alkaline carbonate, (b) at least one polyol, (c) water and/or at least one non-aqueous solvent, and (d) optionally at least one pH adjuster, wherein the at least one alkaline carbonate and the at least one polyol are present in the composition in the form of an associated network. In some embodiments, the molar ratio of alkaline carbonate(s) to polyol(s) in the composition and/or network ranges from less than 1:3 to about 1:50, for example from about 1:4 to about 1:20, from about 1:5 to about 1:15, from about 1:7 to about 1:13, from about 1:9 to about 1:11, or is about 1:10.

In various embodiments, the at least one alkaline carbonate is chosen from sodium carbonate, calcium carbonate, potassium carbonate, magnesium carbonate, lithium carbonate, sodium bicarbonate, potassium bicarbonate, zinc carbonate, or combinations of two or more thereof. In at least some embodiments, the alkaline carbonate(s) comprise, consist essentially of, or consist of potassium carbonate. In various embodiments, the total amount of alkaline carbonates in the composition and/or associated network can range from about 0.1% to about 10%, such as from about 0.25% to about 8%, from about 0.5% to about 6%, from about 0.75% to about 4%, or from about 1% to about 1.5% by weight, relative to the total weight of the composition.

Optionally, the at least one polyol may have a molar mass of less than about 500 g/mol, such as less than about 400 g/mol, less than about 300 g/mol, less than about 200 g/mol, or less than about 150 g/mol. For example, polyols may be chosen from sorbitol, xylitol, maltitol, isomalt, glycerol, and combinations of two or more thereof. In at least some embodiments, the polyol(s) comprise, consist essentially of, or consist of glycerol. In various embodiments, the total amount of polyols in the composition and/or associated network ranges from about 1% to about 25%, such as from about 2% to about 18%, from about 3% to about 15%, from about 4% to about 13%, or from about 5% to about 11% by weight, relative to the total weight of the composition.

In some exemplary embodiments, the compositions comprise (a) at least one alkaline carbonate, preferably including potassium carbonate, (b) at least one polyol, preferably including glycerol, (c) water, (d) at least one pH adjuster, and optionally at least one additional component chosen from active agents and auxiliary components, wherein the at least one alkaline carbonate and the at least one polyol are present in the composition in the form of an associated network, and the molar ratio of alkaline carbonate(s) to polyol(s) in the network ranges from about 1:4 to about 1:20, such as from about 1:5 to about 1:15, from about 1:7 to about 1:13, from about 1:9 to about 1:11, or is about 1:10.

For example, in some embodiments, the compositions comprise (a) at least one alkaline carbonate, (b) at least one polyol, (c) water, (d) at least one pH adjuster, and optionally at least one additional component chosen from active agents and auxiliary components, wherein the alkaline carbonate(s) and polyol(s) are present in the composition in the form of an associated network; the total amount of alkaline carbonate(s) ranges from about 0.1% to about 10%, such as from about 0.25% to about 8%, from about 0.5% to about 5%, or from about 0.75% to about 3%; the total amount of polyol(s) ranges from about 1% to about 25%, such as from about 2% to about 20%, from about 3% to about 15%, or from about 4% to about 12%; the molar ratio of alkaline carbonate(s) to polyol(s) ranges from about 1:4 to about 1:20, such as from about 1:5 to about 1:15, from about 1:7 to about 1:13, from about 1:9 to about 1:11, or is about 1:10; and the pH of the composition is greater than about 7, for example ranges from about 7 to about 14, from about 7 to about 12, from about 7.5 to about 10, or from about 7.5 to about 9.

In further exemplary embodiments, the compositions comprise (a) at least one alkaline carbonate, (b) at least one polyol, (c) water, (d) at least one pH adjuster, and optionally at least one additional component chosen from active agents and auxiliary components, wherein the alkaline carbonate(s) and polyol(s) are present in the composition in the form of an associated network; the total amount of alkaline carbonate(s) ranges from about 4% to about 20%, such as from about 5% to about 18%, from about 6% to about 15%, or from about 8% to about 13%; the total amount of polyol(s) ranges from about 50% to about 95%, such as from about 60% to about 90%, from about 70% to about 85%, or from about 75% to about 80%; the molar ratio of alkaline carbonate(s) to polyol(s) ranges from about 1:4 to about 1:20, such as from about 1:5 to about 1:15, from about 1:7 to about 1:13, from about 1:9 to about 1:11, or is about 1:10; and the pH of the composition is greater than about 7, for example ranges from about 7 to about 14, from about 7 to about 12, from about 7.5 to about 10, or from about 7.5 to about 9.

In still further exemplary embodiments, the compositions comprise (a) potassium carbonate and optionally at least one additional alkaline carbonate, (b) glycerol and optionally at least one additional polyol, (c) water, (d) at least one pH adjuster, and optionally at least one additional component chosen from active agents and auxiliary components, wherein the alkaline carbonate(s) and polyol(s) are present in the composition in the form of an associated network; the total amount of alkaline carbonate(s) ranges from about 0.1% to about 10%, such as from about 0.25% to about 8%, from about 0.5% to about 5%, or from about 0.75% to about 3%; the total amount of polyol(s) ranges from about 1% to about 25%, such as from about 2% to about 20%, from about 3% to about 15%, or from about 4% to about 12%; the molar ratio of alkaline carbonate(s) to polyol(s) ranges from about 1:4 to about 1:20, such as from about 1:5 to about 1:15, from about 1:7 to about 1:13, from about 1:9 to about 1:11, or is about 1:10; and the pH of the composition is greater than about 7, for example ranges from about 7 to about 14, from about 7 to about 12, from about 7.5 to about 10, or from about 7.5 to about 9.

In other exemplary embodiments, the compositions comprise (a) potassium carbonate and optionally at least one additional alkaline carbonate, (b) glycerol and optionally at least one additional polyol, (c) water, and (d) at least one pH adjuster, wherein the alkaline carbonate(s) and polyol(s) are present in the composition in the form of an associated network; the total amount of alkaline carbonate(s) ranges from about 4% to about 20%, such as from about 5% to about 18%, from about 6% to about 15%, or from about 8% to about 13%; the total amount of polyol(s) ranges from about 50% to about 95%, such as from about 60% to about 90%, from about 70% to about 85%, or from about 75% to about 80%; the molar ratio of alkaline carbonate(s) to polyol(s) ranges from about 1:4 to about 1:20, such as from about 1:5 to about 1:15, from about 1:7 to about 1:13, from about 1:9 to about 1:11, or is about 1:10; and the pH of the composition is greater than about 7, for example ranges from about 7 to about 14, from about 7 to about 12, from about 7.5 to about 10, or from about 7.5 to about 9.

The disclosure also relates to methods of treating keratinous substrates such as skin and/or hair using the compositions according to the disclosure, for example for increasing moisture content of the keratinous substrates, providing improved moisture penetration and/or retention, providing conditioning benefits, and/or providing cosmetic benefits such as smoothness, shine, reduction in dryness, etc., to keratinous substrates. In some embodiments, a composition according to the disclosure may be applied to the keratinous substrate, optionally left on the keratinous substrate for a period of time ranging from about 30 seconds to about 48 hours, such as from about 1 minute to about 24 hours, from about 5 minutes to about 12 hours, or from about 10 minutes to about 9 hours, and optionally removing the composition after the leave-on period, for example by rinsing or wiping the keratinous substrates. The keratinous substrates may optionally be cleansed after the leave-on period. For example, in an embodiment where the keratinous substrates are keratinous fibers such as hair, the hair may be cleansed with a shampoo after the leave-on period.

The disclosure also relates to a simplex solution comprising (a) at least one alkaline carbonate, (b) at least one polyol, and (c) optionally water, wherein the at least one alkaline carbonate and the at least one polyol are present in the simplex solution in the form of an associated network. In at least some embodiments, the molar ratio of alkaline carbonate(s) to polyol(s) in the simplex solution ranges from less than 1:3 to about 1:50, for example ranges from about 1:4 to about 1:20, from about 1:5 to about 1:15, from about 1:7 to about 1:13, from about 1:9 to about 1:11, or is about 1:10. In some embodiments where the simplex solution includes water, the molar ratio of alkaline carbonate(s) to water in the simplex solution ranges from about 1:10 to about 10:1, such as from about 5:1 to about 1:5, from about 3:1 to about 1:3, from about 2:1 to about 1:2, or is about 1:1. In some embodiments, the at least one alkaline carbonate is chosen from sodium carbonate, calcium carbonate, potassium carbonate, magnesium carbonate, lithium carbonate, sodium bicarbonate, potassium bicarbonate, zinc carbonate, or combinations of two or more thereof, and at least in some embodiments comprises potassium carbonate. In various embodiments, the at least one polyol has a molar mass of less than about 500 g/mol, less than about 400 g/mol, less than about 300 g/mol, less than about 200 g/mol, or less than about 150 g/mol, for example chosen from sorbitol, xylitol, maltitol, isomalt, glycerol, or combinations of two or more thereof, and at least in some embodiments comprises glycerol.

In various embodiments, the simplex solutions comprise (a) at least one alkaline carbonate, preferably including potassium carbonate, (b) at least one polyol, preferably including glycerol, and (c) optionally water, and the molar ratio of alkaline carbonate(s) to polyol(s) ranges from about 1:4 to about 1:20, such as from about 1:5 to about 1:15, from about 1:7 to about 1:13, from about 1:9 to about 1:11, or is about 1:10.

The disclosure also relates to processes for preparing compositions for treating keratinous substrates, the processes comprising (i) preparing a simplex solution by combining at least one alkaline carbonate, at least one polyol, and optionally water, where the molar ratio of alkaline carbonate(s) to polyol(s) in the simplex solution ranges from less than 1:3 to about 1:50, for example from about 1:4 to about 1:20, from about 1:5 to about 1:15, from about 1:7 to about 1:13, or from about 1:9 to about 1:11, to form an associated network of alkaline carbonate(s) and polyol(s); (ii) optionally heating the simplex solution; (iii) adding at least one solvent chosen from water and/or non-aqueous solvents to the simplex solution to form the composition; and (iv) optionally adding at least one pH adjuster to the composition. The methods in some embodiments comprise heating the simplex solution in (ii) to a temperature ranging from about 35° C. to about 100° C., such as from about 50° C. to about 100° C., or from about 75° C. to about 90° C., and subsequently cooling the simplex solution. The methods in some embodiments comprise adding at least one pH adjuster to the composition to achieve a pH ranging from about 7 to about 14, such as from about 7 to about 11, from about 7.5 to about 10.5, or from about 8 to about 10. The methods in some embodiments comprise adding at least one additional component chosen from active agents and auxiliary components to the composition.

The disclosure also relates to compositions for treating keratinous substrates prepared by the processes of preparing compositions, as well as methods of treating keratinous substrates with compositions prepared by the processes, for example for increasing moisture content of the keratinous substrates, providing improved moisture penetration and/or retention, providing conditioning benefits, and/or providing cosmetic benefits such as smoothness, shine, reduction in dryness, etc., to keratinous substrates.

### BRIEF DESCRIPTION OF THE FIGURES

FIGS. 1A-1B show moisture content data for hair swatches treated with comparative compositions and a composition according to the disclosure.

FIGS. 2A-2B show microscopic analyses of cross sections of hair treated with comparative compositions and a composition according to the disclosure.

### DETAILED DESCRIPTION

The disclosure relates to compositions and methods for treating keratinous substrates, such as skin and hair. The compositions and methods according to the disclosure provide increased moisture and moisture retention to the keratinous substrates, which has surprisingly and unexpectedly been found to be lasting. The disclosure also relates to methods of making the compositions.

### I. Compositions

The compositions according to the present disclosure comprise (a) at least one alkaline carbonate, (b) at least one polyol, (c) water and/or at least one non-aqueous solvent, and (d) optionally at least one pH adjuster. The compositions may optionally comprise at least one active agent and/or at least one auxiliary component.

The alkaline carbonate(s) and polyol(s) are present in the composition in the form of an associated network, and thus, the compositions comprise an associated network of alkaline carbonate(s) and polyol(s). As used herein, the terms “network” and “associated network” are used interchangeably to mean that the components in the composition are associated with one another rather than being present in the composition separately. The association of network components may be chemical or physical. For example, the components may be hydrogen-bonded to one another. However, it is to be understood that compositions according to the disclosure may also comprise one or more of the network components where such component(s) are not part of the network. For example, the compositions may comprise network(s) of alkaline carbonate(s) and polyol(s), and the compositions may comprise one or more alkaline carbonates and/or polyols separate from the network(s).

### Alkaline Carbonates

Compositions according to the disclosure comprise at least one alkaline carbonate. In some embodiments, the compositions and/or associated network may comprise more than one alkaline carbonate, for example at least two alkaline carbonates, at least three alkaline carbonates, etc.

The at least one alkaline carbonate may, in various embodiments, be chosen from alkaline carbonates, alkaline bicarbonates, or combinations thereof. The alkaline carbonates may, for example, be chosen from sodium carbonate, calcium carbonate, potassium carbonate, magnesium carbonate, lithium carbonate, zinc carbonate, or combinations of two or more thereof. The alkaline bicarbonates may, for example, be chosen from sodium bicarbonate, potassium bicarbonate, or combinations thereof. In some embodiments, the alkaline carbonate comprises, consists essentially of, or consists of potassium carbonate.

In various embodiments, total amount of alkaline carbonates in the composition and/or in the associated network ranges from about 0.1% to about 10%, such as from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2.5%, from about 0.1% to about 2%, from about 0.1% to about 1.5%, from about 0.25% to about 10%, from about 0.25% to about 9%, from about 0.25% to about 8%, from about 0.25% to about 7%, from about 0.25% to about 6%, from about 0.25% to about 5%, from about 0.25% to about 4%, from about 0.25% to about 3%, from about 0.25% to about 2.5%, from about 0.25% to about 2%, from about 0.25% to about 1.5%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2.5%, from about 0.5% to about 2%, from about 0.5% to about 1.5%, from about 0.75% to about 10%, from about 0.75% to about 9%, from about 0.75% to about 8%, from about 0.75% to about 7%, from about 0.75% to about 6%, from about 0.75% to about 5%, from about 0.75% to about 4%, from about 0.75% to about 3%, from about 0.75% to about 2.5%, from about 0.75% to about 2%, from about 0.75% to about 1.5%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2.5%, from about 1% to about 2%, from about 1% to about 1.5%, from about 1.25% to about 10%, from about 1.25% to about 9%, from about 1.25% to about 8%, from about 1.25% to about 7%, from about 1.25% to about 6%, from about 1.25% to about 5%, from about 1.25% to about 4%, from about 1.25% to about 3%, from about 1.25% to about 2.5%, from about 1.25% to about 2%, or from about 1.25% to about 1.5% by weight, relative to the total weight of the composition. In some embodiments, the total amount of alkaline carbonates in the composition and/or in the associated network ranges from about 0.2% to about 2.5%, such as from about 0.3% to about 2.4%, from about 0.4% to about 2.3%, from about 0.5% to about 2.2%, from about 0.6% to about 2.1%, from about 0.7% to about 2%, from about 0.8% to about 1.9%, from about 0.9% to about 1.8%, from about 1% to about 1.7%, or from about 1.1% to about 1.6% by weight, relative to the total weight of the composition. In some embodiments, the total amount of alkaline carbonates in the composition and/or in the associated network may range from about 0.5% to about 2.5%, such as from about 0.75% to about 1.75%, or from about 1% to about 1.5% by weight, relative to the total weight of the composition.

In other embodiments, the total amount of alkaline carbonates in the composition and/or in the associated network may range from about 4% to about 20%, from about 4% to about 18%, from about 4% to about 16%, from about 4% to about 14%, from about 4% to about 12%, from about 4% to about 10%, from about 5% to about 20%, from about 5% to about 18%, from about 5% to about 16%, from about 5% to about 14%, from about 5% to about 12%, from about 5% to about 10%, from about 6% to about 20%, from about 6% to about 18%, from about 6% to about 16%, from about 6% to about 14%, from about 6% to about 12%, from about 6% to about 10%, from about 7% to about 20%, from about 7% to about 18%, from about 7% to about 16%, from about 7% to about 14%, from about 7% to about 12%, from about 7% to about 10%, from about 8% to about 20%, from about 8% to about 18%, from about 8% to about 16%, from about 8% to about 14%, from about 8% to about 12%, from about 8% to about 10%, from about 9% to about 20%, from about 9% to about 18%, from about 9% to about 16%, from about 9% to about 14%, from about 9% to about 12%, from about 9% to about 10%, or may be about 8%, about 9%, about 10%, about 11%, about 12%, about 13%, or about 14% by weight, relative to the total weight of the compositions. For example, in some embodiments, the total amount of alkaline carbonates ranges from about 5% to about 18%, such as from about 6% to about 15%, or from about 8% to about 13% by weight, relative to the total weight of the composition.

### Polyols

Compositions according to the disclosure comprise at least one polyol. In some embodiments, the compositions and/or associated network may comprise more than one polyol, for example at least two polyols, at least three polyols, etc.

The term “polyol,” as used herein, refers to an organic molecule comprising at least two hydroxyl groups, for example, at least three hydroxyl groups, such as trehalose. In various embodiments, the polyols may have from 2 to 32 carbon atoms, such as from 3 to 16 carbon atoms, or from 3 to 12 carbon atoms. Optionally, the polyols are chosen from alkane polyols. In some embodiments, the polyols are not chosen from sugar alcohols or derivatives thereof.

In various embodiments, polyols that can be chosen include polyols with a molar mass of less than about 500 g/mol, for example less than about 450 g/mol, less than about 400 g/mol, less than about 350 g/mol, less than about 300 g/mol, less than about 250 g/mol, less than about 200 g/mol, less than about 175 g/mol, less than about 150 g/mol, less than about 125 g/mol, or less than about 100 g/mol.

By way of example only, polyols that may be chosen include glycerol (glycerin), sorbitol, xylitol, maltitol, isomalt, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, 4-methyl-1,2-pentanediol, or a mixture of two or more thereof. In some embodiments, the polyol comprises, consists essentially of, or consists of glycerol.

In various embodiments, the total amount of polyols present in the composition and/or in the associated network ranges from about 1% to about 25%, such as from about 1% to about 20%, from about 1% to about 15%, from about 1% to about 14%, from about 1% to about 13%, from about 1% to about 12%, from about 1% to about 11%, from about 1% to about 10%, from about 1% to about 9%, from about 1% to about 8%, from about 1% to about 7%, from about 1% to about 6%, from about 1% to about 5%, from about 2% to about 25%, from about 2% to about 20%, from about 2% to about 15%, from about 2% to about 14%, from about 2% to about 13%, from about 2% to about 12%, from about 2% to about 11%, from about 2% to about 10%, from about 2% to about 9%, from about 2% to about 8%, from about 2% to about 7%, from about 2% to about 6%, from about 2% to about 5%, from about 3% to about 25%, from about 3% to about 20%, from about 3% to about 15%, from about 3% to about 14%, from about 3% to about 13%, from about 3% to about 12%, from about 3% to about 11%, from about 3% to about 10%, from about 3% to about 9%, from about 3% to about 8%, from about 3% to about 7%, from about 3% to about 6%, from about 3% to about 5%, from about 4% to about 25%, from about 4% to about 20%, from about 4% to about 15%, from about 4% to about 14%, from about 4% to about 13%, from about 4% to about 12%, from about 4% to about 11%, from about 4% to about 10%, from about 4% to about 9%, from about 4% to about 8%, from about 4% to about 7%, from about 4% to about 6%, from about 4% to about 5%, from about 5% to about 25%, from about 5% to about 20%, from about 5% to about 15%, from about 5% to about 14%, from about 5% to about 13%, from about 5% to about 12%, from about 5% to about 11%, from about 5% to about 10%, from about 5% to about 9%, from about 5% to about 8%, from about 5% to about 7%, or from about 5% to about 6% by weight, relative to the total weight of the composition. In some embodiments, the total amount of polyols in the composition and/or in the associated network ranges from about 5% to about 12%, such as from about 5.5% to about 11%, from about 6% to about 10%, from about 6.5% to about 9.5%, from about 7% to about 9%, or from about 8% to about 9% by weight, relative to the total weight of the composition.

In other embodiments, the total amount of polyols ranges from about 50% to about 95%, such as from about 55% to about 90%, from about 55% to about 85%, from about 55% to about 80%, from about 55% to about 75%, from about 55% to about 70%, from about 60% to about 90%, from about 60% to about 85%, from about 60% to about 80%, from about 60% to about 75%, from about 60% to about 70%, from about 65% to about 90%, from about 65% to about 85%, from about 65% to about 80%, from about 65% to about 75%, from about 65% to about 70%, from about 70% to about 90%, from about 70% to about 85%, from about 70% to about 80%, from about 70% to about 75%, from about 75% to about 90%, from about 75% to about 85%, from about 75% to about 80% by weight, relative to the total weight of the composition. In some embodiments, the total amount of polyols may range from about 60% to about 90%, from about 70% to about 85%, or from about 75% to about 80% by weight, relative to the total weight of the composition.

It may be particularly advantageous, in certain embodiments, to choose amounts of alkaline carbonates and polyols such that the molar ratio of the alkaline carbonate(s) to polyol(s) present in the composition and/or in the associated network is less than 1:3 and greater than about 1:50, for example ranges from about 1:4 to about 1:50, from about 1:4 to about 1:45, from about 1:4 to about 1:40, from about 1:4 to about 1:35, from about 1:4 to about 1:30, from about 1:4 to about 1:25, from about 1:4 to about 1:20, from about 1:4 to about 1:15, from about 1:4 to about 1:10, from about 1:5 to about 1:50, from about 1:5 to about 1:45, from about 1:5 to about 1:40, from about 1:5 to about 1:35, from about 1:5 to about 1:30, from about 1:5 to about 1:25, from about 1:5 to about 1:20, from about 1:5 to about 1:15, or from about 1:5 to about 1:10. In some embodiments, the molar ratio of alkaline carbonate(s) to polyol(s) may range from about 1:4 to about 1:20, from about 1:5 to about 1:15, from about 1:7 to about 1:13, or from about 1:9 to about 1:11, for example a molar ratio of about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, or about 1:20, or may be in a range having any of the foregoing as upper and lower endpoints.

### pH Adjusters

Compositions according to the disclosure have a pH of greater than or equal to 7, such as from about 7 to about 14, from about 7 to about 13, from about 7 to about 12, from about 7 to about 11, from about 7 to about 10, from about 7 to about 9, from about 7.5 to about 14, from about 7.5 to about 13, from about 7.5 to about 12, from about 7.5 to about 11, from about 7.5 to about 10, or from about 7.5 to about 9, such as, for example, from about 7.5 to about 10.5, or from about 8 to about 10. For example, the pH of the composition may be about 7.5, about 8, about 8.5, about 9, about 9.5, about 10, about 10.5, about 11, about 11.5, about 12, about 12.5, about 13, about 13.5, or about 14, or may be in a range having any of the foregoing as upper and lower endpoints. Therefore, if needed, compositions according to the disclosure may optionally comprise at least one pH adjuster. In various embodiments, the pH adjuster is chosen from compounds that can lower the pH of the composition, for example inorganic or organic acids which are commonly used such as citric acid, lactic acid, maleic acid, malonic acid, hydrochloric acid, etc.

### Solvents

Compositions according to the present disclosure comprise at least one solvent chosen from water and/or non-aqueous solvents. In various embodiments, the compositions comprise water in an amount ranging from about 60% to about 98%, such as from about 70% to about 95% or about 80% to about 90% by weight, relative to the total weight of the composition.

Optionally, compositions according to the disclosure comprise at least one non-aqueous solvent. If present, the non-aqueous solvent(s) may be chosen from, for example, monoalcohols, polyols other than those described above, fatty alcohols, fatty ethers, fatty esters, glycols, vegetable oils, mineral oils, silicone oils, liposomes, laminar lipid materials, or any mixture thereof.

### Active Agents

Optionally, the compositions can comprise one or more active agents. In some embodiments, the compositions comprise at least two active agents, at least three active agents, etc.

By way of non-limiting example, the active agents may be chosen from amino acids, amino sulfonic acids, and salts thereof. The amino acids, amino sulfonic acids, and salts thereof may be chosen from acidic, basic, or neutral amino acids, amino sulfonic acids, and salts thereof. For example, one or more of alanine, arginine, asparagine, aspartic acid, cysteine, glutamic acid, glutamine, glycine, histidine, isoleucine, leucine, lysine, methionine, phenylalanine, proline, serine, taurine, threonine, tryptophan, tyrosine, valine, or salts thereof may be included. By way of non-limiting example only, the compositions may comprise one or more of betaine, taurine, glycine, or salts thereof; one or more salts of non-acidic amino acids; or combinations of two or more thereof.

As further non-limiting examples, vitamins and derivatives or prodrugs of vitamins, as well as their salts, may be included. By way of example only, the compositions may comprise panthenol, tocopherol, ascorbic acid, salts thereof, or combinations thereof.

As yet further examples, phenolic and polyphenolic compounds may be included. For example, lignans, humates, flavones, chalcones, tannins, phenolic acids, catechins, anthocyanidins, stilbenoids, curcuminoids, or phenylpropanenoids may be chosen. Some non-limiting examples are baicalin, resveratrol, ferulic acid, ellagic acid, and salicylic acid.

Still further examples of optional active agents include salts of acids (e.g. sodium citrate and/or sodium lactate), ceramides, cholesterols, sphingosines, C-glycosides, sugars (e.g. monosaccharides such as glucose, fructose, and/or pentose, disaccharides such as sucrose, maltose, and/or lactose, etc.), nucleic acids, α- and β-hydroxy acids, botanical extracts, silicates, aminopropyl triethoxysilane (APTES), dihydroxyacetone (DHA), peptides, derivatives thereof, and mixtures thereof.

In some embodiments, the compositions comprise one or more active agents chosen from vitamins, derivatives of vitamins, prodrugs of vitamins, sol-gel agents, peptides, amino acids or salts thereof, amino sulfonic acids or salts thereof, salts of acids, polyphenolic compounds, moisturizing agents, or combinations of two or more thereof.

If present, the total amount of active agent(s) and/or the amount of individual active agents may range from about 0.001% to about 10%, such as from about 0.001% to about 9%, from about 0.001% to about 8%, from about 0.001% to about 7%, from about 0.001% to about 6%, from about 0.001% to about 5%, from about 0.001% to about 4%, from about 0.001% to about 3%, from about 0.001% to about 2%, from about 0.001% to about 1%, from about 0.001% to about 0.5%, from about 0.001% to about 0.1%, from about 0.01% to about 10%, from about 0.01% to about 9%, from about 0.01% to about 8%, from about 0.01% to about 7%, from about 0.01% to about 6%, from about 0.01% to about 5%, from about 0.01% to about 4%, from about 0.01% to about 3%, from about 0.01% to about 2%, from about 0.01% to about 1%, from about 0.01% to about 0.5%, from about 0.01% to about 0.1%, from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, or from about 0.1% to about 0.5% by weight, relative to the total weight of the composition. For example, in various embodiments, the total amount of active agent(s) and/or the amount of individual active agents may be less than about 10%, less than about 9%, less than about 8%, less than about 7%, less than about 6%, less than about 5%, less than about 4%, less than about 3%, less than about 2%, or less than about 1% by weight, relative to the total weight of the composition. In some embodiments, the total amount of active agents and/or the amount of individual active agents may range from about 0.001% to about 5%, such as from about 0.001% to about 3%, from about 0.005% to about 5%, about 0.005% to about 3%, from about 0.01% to about 5%, or from about 0.01% to about 3% by weight, relative to the total weight of the composition.

### Auxiliary Components

Compositions according to the disclosure may optionally include one or more auxiliary components. Useful auxiliary components include, but are not limited to, clays (e.g. kaolinite, bentonite, hectorite, smectite, etc.), dyes and/or pigments for coloring the composition, rheology modifying agents (thickeners), humectants, structuring agents, shine agents, antioxidants, penetrants, sequestrants, fragrances, opacifiers, sunscreens, etc.

Optional auxiliary components may be present in an amount ranging up to about 15% by weight, such as from about 0.01% to about 10%, or about 0.1% to about 5% by weight, relative to the total weight of the composition.

Compositions according to the disclosure may be in any form, such as a serum, an emulsion, a gel, a liquid, etc., and may be leave-in or rinse-out compositions.

### II. Methods

The disclosure also relates to methods of treating keratinous substrates with compositions according to the disclosure. For example, the methods may comprise methods of increasing or improving moisture in keratinous substrates, methods of reducing moisture loss in keratinous substrates, methods of conditioning keratinous substrates, etc. The methods may also be methods of treating hair with compositions according to the disclosure, for example by increasing or improving moisture in the hair fibers, reducing moisture loss from the hair fibers, conditioning the hair such as by improving smoothness, discipline (e.g. less fly-aways), and strength of hair, reducing frizz, and/or improving curl definition and/or curl regularity of curly hair.

Typically, the methods of treating keratinous substrates with compositions according to the disclosure comprise applying the composition to the keratinous substrates, and optionally removing the composition after a period of time, for example by rinsing or wiping the composition from the keratinous substrate. The period of time that the composition is left on the keratinous substrate (“resting period” or “leave-on period”) can vary, but may range from about 30 seconds to about 48 hours, such as from about 30 seconds to about 24 hours, from about 30 seconds to about 12 hours, from about 30 seconds to about 6 hours, from about 30 seconds to about 2 hours, from about 30 seconds to about 1 hour, from about 30 seconds to about 45 minutes, from about 30 seconds to about 30 minutes, from about 30 seconds to about 20 minutes, from about 30 seconds to about 15 minutes, from about 30 seconds to about 10 minutes, from about 30 seconds to about 5 minutes, or from about 30 seconds to about 2 minutes. For example, in some embodiments, the resting period can range from about 1 minute to about 2 hours, from about 5 minutes to about 1 hour, from about 10 minutes to about 45 minutes, or from about 15 minutes to about 40 minutes, such as about 30 minutes. In other embodiments, the resting period can range from about 1 hour to about 24 hours, from about 2 hours to about 12 hours, or from about 3 hours to about 9 hours, e.g. the composition may be left on the keratinous substrate overnight.

Optionally, the composition may be applied to the keratinous substrate and left on the keratinous substrate until the keratinous substrate is next washed in the ordinary course of personal grooming. As such, in some embodiments the composition may be considered a “pre-wash” or “pre-shampoo” treatment composition. For example, one exemplary and non-limiting method according to the disclosure comprises applying a composition according to the disclosure to the skin or hair and left for a leave-on period, for example for a period of time ranging from about 1 minute to about 24 hours, such as from about 5 minutes to about 12 hours, or from about 15 minutes to about 9 hours, for example overnight, or from about 1 minute to about 2 hours, from about 5 minutes to about 1 hour, from about 10 minutes to about 45 minutes, or from about 15 minutes to about 40 minutes, such as about 30 minutes, and subsequently washing the skin or hair.

Surprisingly, the compositions and methods of treating keratinous substrates according to the disclosure have been found to be exceedingly effective at not only increasing the moisture content of the keratinous substrates, but also at maintaining the moisture content of the keratinous substrates, e.g. by reducing the amount of moisture lost from the keratinous substrates, which is believed to be a result of an unexpected synergistic interaction of the components described herein. Additionally, although the compositions are alkaline, the compositions are not damaging to the keratinous substrates, e.g. keratin fibers such as hair, which is also believed to be a result of the synergistic interaction of the components described herein.

Without being bound by theory, it is believed that the alkaline carbonate(s) and polyol(s) form a unique network that permits increased penetration into the keratinous substrates, and also remains within the keratinous substrates and maintains an ideal level of moisture within the keratinous substrates that lasts even under conditions such as high or low humidity that would otherwise result in water entering or exiting the keratinous substrates. Thus, the network reduces the influence of the external environment on the moisture content of the keratinous substrate, permitting the optimum moisture and/or hydration level within the keratin materials to be maintained.

Methods of making compositions according to the disclosure thus comprise preparing an associated network of alkaline carbonate(s) and polyol(s). For example, a simplex solution of alkaline carbonate(s) and polyol(s) may be prepared, which may include added water but is preferably prepared with a minimal amount of added water or even no added water. Without being limited by theory, it is believed that by preparing a simplex solution as described, the alkaline carbonate(s) and polyol(s) form a hydrogen-bonded network.

For example, a simplex solution of alkaline carbonate(s) and polyol(s) may be prepared by combining the alkaline carbonate(s), polyol(s), and optionally a small amount of water and stirring until the alkaline carbonate dissolves and the network is formed. In various embodiments, the amount of water in the simplex solution may range up to about 10%, for example may range from about 0.01% to about 10%, from about 0.01% to about 5%, from about 0.01% to about 3%, from about 0.01% to about 2%, from about 0.01% to about 1%, from about 0.1% to about 10%, from about 0.1% to about 5%, from about 0.1% to about 3%, from about 0.1% to about 2%, or from about 0.1% to about 1% by weight, based on the total weight of the simplex solution. In some embodiments, no water is added to the simplex solution. Optionally, one or more of the active agents described above may also be included in the simplex solution, and the active agent(s) may thus also be present in the network.

The process may optionally take place at room temperature or may be elevated, for example, may range from about 22° C. to about 25ºC, from about 25° C. to about 100° C., from about 35° C. to about 100° C., from about 50° C. to about 100° C., from about 75° C. to about 100° C., from about 25° C. to about 90ºC, from about 35° C. to about 90° C., from about 50° ° C. to about 90° C., or from about 75° C. to about 90° C.

Preferably, the simplex solution is prepared by including amounts of alkaline carbonate(s) and polyol(s) such that the molar ratio of the alkaline carbonate(s) to polyol(s) present in the simplex solution, and thus the network, is less than 1:3 to greater than about 1:50, for example ranges from about 1:4 to about 1:50, from about 1:4 to about 1:45, from about 1:4 to about 1:40, from about 1:4 to about 1:35, from about 1:4 to about 1:30, from about 1:4 to about 1:25, from about 1:4 to about 1:20, from about 1:4 to about 1:15, from about 1:4 to about 1:10, from about 1:5 to about 1:50, from about 1:5 to about 1:45, from about 1:5 to about 1:40, from about 1:5 to about 1:35, from about 1:5 to about 1:30, from about 1:5 to about 1:25, from about 1:5 to about 1:20, from about 1:5 to about 1:15, or from about 1:5 to about 1:10. For example, in some embodiments, the molar ratio of alkaline carbonate(s) to polyol(s) in the simplex solution may range from about 1:4 to about 1:20, from about 1:5 to about 1:15, from about 1:7 to about 1:13, or from about 1:9 to about 1:11, for example a molar ratio of about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, or about 1:20, or may be in a range using any of the foregoing as upper and lower endpoints. In some embodiments, the molar ratio of alkaline carbonate(s) to polyol(s) in the simplex solution is about 1:10.

In some embodiments, the simplex solution comprises water and the molar ratio of the alkaline carbonate(s) to water ranges from about 10:1 to about 1:10, such as from about 5:1 to about 1:5, for example from about 4:1 to about 1:4, about 3:1 to about 1:3, about 2.5:1 to about 1:2.5, about 1:2 to about 2:1, about 1.75:1 to about 1:1.75, about 1.5:1 to about 1:1.5, about 1.25:1 to about 1:1.25, about 1.2:1 to about 1:1.2, or about 1.1:1 to about 1:1.1. For example, the molar ratio of alkaline carbonate(s) to water may be about 5, about 4.5, about 4, about 3.5, about 3, about 2.5, about 2, about 1.9, about 1.8, about 1.7, about 1.6, about 1.5, about 1.4, about 1.3, about 1.2, about 1.1, about 1, about 0.9, about 0.8, about 0.7, about 0.6, or about 0.5, or may be in a range using any of the foregoing as upper and lower endpoints. Optionally, if present, the amount of water in the simplex solution is such that the molar ratio of alkaline carbonate(s) to water is approximately the same, i.e. is approximately 1:1.

Once the network of alkaline carbonate(s) and polyol(s) is formed, the network can be included in the compositions according to the disclosure as described above. For example, a simplex solution of alkaline carbonate(s) and polyol(s) may be prepared as described herein, and the simplex solution may then be added to or diluted with water and/or a non-aqueous solvent to form a composition according to the disclosure, and the pH of the composition may optionally be adjusted to the target pH by adding a pH adjuster. More than one network of alkaline carbonate(s) and polyol(s) may be included in the compositions, wherein the alkaline carbonate(s) and/or polyol(s) in each network can be the same or different from those in the other network(s). Additional components such as one or more active agents and/or auxiliary components may optionally also be included in the compositions. However, a skilled artisan will take care not to include components or amounts of components in the simplex solution and/or composition that could diminish the network interaction of the alkaline carbonate(s) and polyol(s).

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” and “at least one” are interchangeable and expressly include individual components as well as mixtures/combinations. Likewise, the term “a salt thereof” also relates to “salts thereof.” Thus, where the disclosure refers to “at least one element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two or more of A, B, C, D, E, F, one or more salts of A, one or more salts of B, one or more salts of C, one or more salts of D, one or more salts of E, and one or more salts of F may be included.

The term “and/or” should be understood to include both the conjunctive and the disjunctive. For example, “amino acids and/or salts thereof” means “amino acids and salts thereof” as well as “amino acids or salts thereof,” and expressly covers instances of either.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Unless expressly stated otherwise, “about” means +/−5%. Likewise, all endpoints of ranges are understood to be individually disclosed, such that, for example, a range of 1:2 to 2:1 is understood to disclose a ratio of both 1:2 and 2:1.

As used herein, if a component is described as being present “in an amount up to” a certain amount, it is intended that such component is, in fact, present in the composition, i.e. is present in an amount greater than 0%.

All amounts and ratios herein are given based upon the total weight of the composition, unless otherwise indicated. Unless otherwise indicated, all percentages herein are by weight of active material.

As used herein, the terms “applying a composition onto keratin substrates” variations thereof are intended to mean contacting the keratin substrates such as hair and skin, with at least one of the compositions of the disclosure, in any manner. It may also mean contacting the keratin substrates with an effective amount of the composition.

As used herein, the term “salts” referred to throughout the disclosure may include salts having a counterion such as an alkali metal, alkaline earth metal, or ammonium counterion. This list of counterions, however, is non-limiting. Salts also include a dissociated form of a compound, e.g. in an aqueous solution.

As used herein, the term “substantially free” or “essentially free” means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “treat” (and its grammatical variations) refers to the application of the compositions of the present disclosure onto the surface of keratin substrates, such as the skin and/or hair.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following Examples are intended to be non-limiting and explanatory in nature only. In the Examples, amounts are expressed in percentage by weight (wt %) of active materials, relative to the total weight of the composition, unless otherwise indicated.

### Example 1—Simplex Solutions

The following three simplex solutions were prepared to form three different associated networks of potassium carbonate and glycerol.

### Example 1A—Simplex Solution With Water

Potassium carbonate, glycerol, and deionized water were mixed at a molar ratio of about 1:10:1 while stirring at a temperature of about 80° C. until the potassium carbonate dissolved and the solution was substantially homogeneous. The solution was allowed to cool to room temperature. It was observed that the solution was highly viscous.

### Example 1B—Simplex Solutions Without Water

Two separate simplex solutions were prepared. Potassium carbonate and glycerol were mixed at a molar ratio of about 1:10 while stirring at a temperature of about 80° C. until the potassium carbonate dissolved and the solution was substantially homogeneous. The solution was allowed to cool to room temperature. The procedure was repeated but the molar ratio of potassium carbonate to glycerol was about 1:5. In both procedures it was observed that the solutions were highly viscous.

### Example 2—Compositions

The inventive and comparative compositions in Table 1 were prepared.

TABLE 1

| | Inventive | | | | Comparative | | | | |
|---|---|---|---|---|---|---|---|---|---|
| | 2A | 2B | 2C | 2D | C1 | C2 | C3 | C4 | C5 |
| Glycerol | 8.6 | 8.6 | 77.4 | | 8.6 | 8.6 | 77.4 | | |
| Deuterated | | | | 8.6 | | | | 8.6 | 8.6 |
| glycerol | | | | | | | | | |
| Potassium | 1.3 | 1.3 | 11.7 | 1.3 | | 1.3 | | | 1.3 |
| carbonate | | | | | | | | | |
| Hydrochloric | QS to | | | | | | | | |
| acid | pH 8 | | | | | | | | |
| Lactic acid | | QS to | QS to | QS to | | QS to | | | QS to |
| | | pH 8 | pH 8 | pH 8 | | pH 8 | | | pH 8 |
| Water | QS to | QS to | QS to | QS to | QS to | QS to | QS to | QS to | QS to |
| | 100 | 100 | 100 | 100 | 100 | 100 | 100 | 100 | 100 |
| pH | 8 | 8 | 8 | 8 | 7 | 8 | 7 | 7 | 8 |

Compositions 2A-2C according to the disclosure were prepared by diluting the simplex solution prepared in Example 1A with water and pH adjuster. Composition 2D was prepared in the same manner as the simplex solution prepared in Example 1A, but using deuterated glycerol, and then diluted with water and pH adjuster.

Compositions C1 and C3 were prepared by mixing water and glycerol, and composition C4 was prepared by mixing water and deuterated glycerol. The pH of the mixtures were measured to be 7.

Compositions C2 and C5 were prepared by mixing all the specified components together at once to prepare the composition in the form of an admixture of potassium carbonate and glycerol in water.

### Example 3—Expert Testing

The following tests were performed to evaluate the moisture and cosmetic benefits imparted to hair by inventive and comparative compositions in Table 1.

### Example 3A-1

Two swatches of very curly hair were rinsed, shampooed, rinsed again, and left to air dry overnight under ambient conditions. Equal amounts (approximately 0.4 grams per gram of hair) of either composition 2A or C1 were applied to separate swatches and massaged into the hair. The swatches were left over night (approximately 12 hours) under ambient conditions. Subsequently, the swatches were shampooed, rinsed, and left to air dry overnight under ambient conditions.

Consistent with how such properties are typically evaluated in the art, an expert evaluated the visual and tactile properties of each swatch to determine whether various properties of the swatch treated with inventive composition 2A or the swatch treated with comparative composition C1 were better. The expert concluded that the swatch treated with inventive composition 2A demonstrated noticeably better moisturization and curl regularity and curl definition, and slightly better smoothness and frizz reduction than the swatch treated with comparative composition C1.

### Example 3A-2

Either composition 2A or C1 was applied to dry, curly hair on two mannequin heads with hair divided into four quadrants, with each quadrant having a different curl pattern. The compositions remained on the hair for a leave-on period of about 30 minutes, after which the hair was shampooed and rinsed, a detangling solution was applied and subsequently the hair was rinsed again, and the hair was then dried under a rollerball dryer for about one hour.

Consistent with how such properties are typically evaluated in the art, an expert evaluated the visual and tactile properties of each head of hair to determine whether various properties of the hair treated with inventive composition 2A or with comparative composition C1 were better. The expert evaluated three of the four quadrants on each mannequin head, the least curly quadrant and the two most curly quadrants, for a total of 3 evaluations for the hair treated with inventive composition 2A and 3 evaluations for the hair treated with comparative composition C1. Each quadrant of hair on the mannequin head treated with inventive composition 2A was assigned a rating of 1 (slightly better), 2 (noticeably better), or 3 (dramatically better) to quantify the improvement of the parameter compared to the corresponding quadrant of hair on the mannequin head treated with comparative composition C1. The average (n=3) of these ratings is set forth in Table 2 below.

TABLE 2

| Parameter | Improvement Rating |
|---|---|
| Dryness reduction (tactile) | 2.3 |
| Shape control | 2.3 |
| Discipline | 2 |
| Moisturization | 2 |
| Smoothness (tactile) | 2 |
| Smoothness (visual) | 1.7 |
| Reduction in dry ends (visual) | 1.7 |
| Strength | 1.7 |

The expert also observed that the two quadrants of the most curly hair treated with inventive composition 2A had significantly better detangling effects on application than the same quadrants of hair treated with comparative composition C1.

Examples 3A-1 and 3A-2 therefore confirm that compositions according to the disclosure comprising an associated network of alkaline carbonate(s) and polyol(s) surprisingly provide noticeable moisture, conditioning, and cosmetic benefits to hair compared to compositions comprising a polyol but no alkaline carbonate.

### Example 3B-1

Half-head studies were conducted on dry, curly hair of six volunteers. For each volunteer, inventive composition 2B was applied to one half of the head of hair and comparative composition C2 was applied to the other half of the head of hair. The compositions were applied and massaged through the hair to evenly distribute the compositions, and left on the hair for 30 minutes, after which the hair was shampooed and rinsed, a detangling solution was applied and subsequently the hair was rinsed again, and the hair was then dried under a rollerball dryer for about one hour.

Consistent with how such properties are typically evaluated in the art, an expert evaluated the visual and tactile properties of each half head of hair to determine whether various properties of the hair treated with inventive composition 2B or the hair treated with comparative composition C2 were better. Each half head of hair treated with inventive composition 2B was assigned a rating of 1 (slightly better), 2 (noticeably better), or 3 (dramatically better) to quantify the improvement of the parameter compared to the hair treated with comparative composition C2. The average (n=6) of these ratings is set forth in Table 3 below.

TABLE 3-1

| Parameter | Improvement Rating |
|---|---|
| Smoothness (tactile) | 1.7 |
| Curl definition | 1.5 |
| Moisturization | 1.5 |
| Strength | 1.5 |
| Dryness reduction (tactile) | 1.5 |

The expert also observed that volunteers with the curliest hair (n=4) had significantly better curl resiliency on the half of the head treated with inventive composition 2B compared to the half of the head treated with comparative composition C2.

### Example 3B-2

Half-head studies were conducted on dry, curly hair of three volunteers. For each volunteer (n=3), inventive composition 2B was applied to one half of the head of hair and deionized water was applied to the other half of the head of hair. Composition 2B or the water was applied and massaged through the hair to evenly distribute the composition or water, and left on the hair for 30 minutes. After 30 minutes the hair was very gently wet, and washed using a commercially-available shampoo and conditioner bundle and subsequently the hair was rinsed again, and the hair was then dried under a rollerball dryer for about one hour.

Consistent with how such properties are typically evaluated in the art, an expert evaluated the visual and tactile properties of each half head of hair to determine whether various properties of the hair treated with inventive composition 2B or the hair treated with deionized water were better. Each half head of hair treated with inventive composition 2B was assigned a rating of 1 (slightly better), 2 (noticeably better), or 3 (dramatically better) to quantify the improvement of the parameter compared to the hair treated with deionized water. The average (n=3) of these ratings is set forth in Table 3-2 below.

TABLE 3-2

| | Day of | Day After |
|---|---|---|
| Parameter | Application | Application |
| Ease of detangling (during S + C) | 2 | 2 |
| Moisturization | 1 | 1.3 |
| Discipline | 1 | 1.7 |
| Dryness reduction (tactile) | 1.7 | 1.3 |
| Dryness reduction (visual) | 1 | 1.7 |
| Smoothness (visual) | 1.3 | 1.3 |
| Smoothness (tactile) | 1 | 1.3 |
| Curl elongation | 1.3 | 1.3 |

### Example 3B-3

Half-head studies were conducted on dry, curly, unwashed hair of three volunteers. For each volunteer (n=3), inventive composition 2B was applied to one half of the head of hair and deionized water was applied to the other half of the head of hair. Composition 2B or the water was applied and massaged through the hair to evenly distribute the composition or water, and left on the hair for 30 minutes. After 30 minutes the hair was very gently wet and washed using a different commercially-available shampoo and conditioner bundle than in Example 3B-2, and the hair was then dried under a rollerball dryer for about one hour.

Consistent with how such properties are typically evaluated in the art, an expert evaluated the visual and tactile properties of each half head of hair to determine whether various properties of the hair treated with inventive composition 2B or the hair treated with deionized water were better. Each half head of hair treated with inventive composition 2B was assigned a rating of 1 (slightly better), 2 (noticeably better), or 3 (dramatically better) to quantify the improvement of the parameter compared to the hair treated with deionized water. The average (n=3) of these ratings is set forth in Table 3-3 below.

TABLE 3-3

| | Day of | Day After |
|---|---|---|
| Parameter | Application | Application |
| Moisturization | 1 | 2 |
| Dryness reduction (visual) | 1.7 | 1.7 |
| Curl regularity | 1.7 | 2 |
| Dryness reduction (tactile) | 1.7 | 2 |
| Smoothness (tactile) | 1.7 | 1.7 |
| Smoothness (visual) | 1.3 | 1.7 |
| Discipline | 1 | 1.7 |

Examples 3B-1, 3B-2, and 3B-3 therefore demonstrate that compositions according to the disclosure comprising an associated network of alkaline carbonate(s) and polyol(s) surprisingly provide noticeable moisture, conditioning, and cosmetic benefits to hair compared to compositions comprising the same components but not as an associated network.

### Example 4—Instrumental Testing

The following tests were performed to evaluate the moisture benefits imparted to hair by inventive and comparative compositions in Table 1.

### Example 4A—Microwave Analysis

Six swatches of hair (curly (n=3) and very curly (n=3)) were rinsed, shampooed with a commercially-available shampoo, rinsed again, treated with a commercially-available detangler, combed, rinsed, and left to air dry overnight under ambient conditions. Equal amounts (approximately 0.15 grams per gram of hair) of one of compositions 2C, C3, or deionized water were applied to separate swatches curly and very curly hair, and massaged into the hair. The swatches were then placed in a humidity chamber (RH=45%).

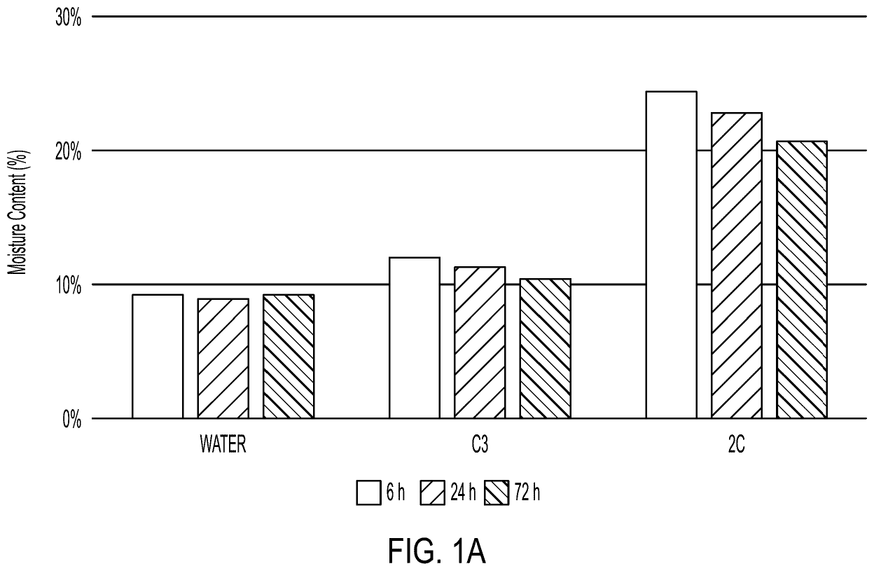

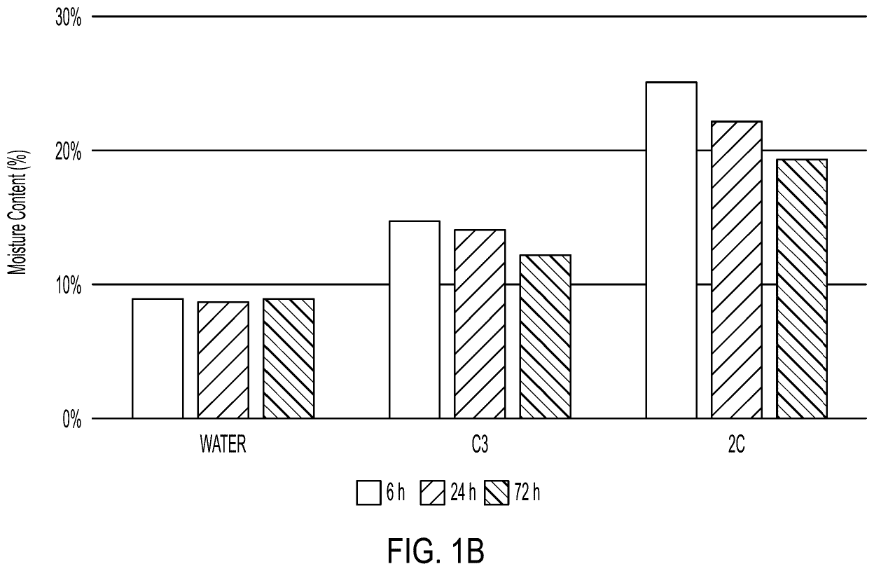

The moisture content of each of the hair swatches was evaluated using a TEWS microwave resonance device at various time points (6 hours, 24 hours, and 72 hours) after the swatches were placed into the humidity chamber. FIGS. 1A (curly) and 1B (very curly) show the moisture content for hair swatches treated with compositions 2C, C3, or deionized water. As FIGS. 1A-1B demonstrate, at all time points the hair treated with composition 2C according to the disclosure surprisingly attracted and retained a significantly higher amount of moisture than hair treated with either composition C3 or water.

### Example 4B—Attenuated Total Reflection Fourier Transform Infrared Microscopy

Seven swatches of hair (n=7) were rinsed, shampooed with a commercially-available shampoo, rinsed again, treated with a commercially-available detangler, combed, rinsed, and left to air dry overnight under ambient conditions. Equal amounts (approximately 1 gram per gram of hair) of one of compositions 2D, C4, C5, or deionized water (control) were applied to separate swatches (labelled A-G) and massaged into the hair. The treated swatches were then left to rest under ambient conditions for 30 minutes. The swatches were then again rinsed, shampooed with a commercially-available shampoo, rinsed, treated with a commercially-available detangler, combed, rinsed, and left to air dry under ambient conditions. This process constituted one (1) treatment.

Three of the swatches, E, F, and G, were subjected to the above process four additional times, for a total of five (5) treatments. Table 4 correlates compositions and number of treatments with each swatch.

TABLE 4

| Swatch | A | B | C | D | E | F | G |
|---|---|---|---|---|---|---|---|
| Composition | water | C4 | C5 | 2D | C4 | C5 | 2D |
| Number of Treatments | — | 1 | 1 | 1 | 5 | 5 | 5 |

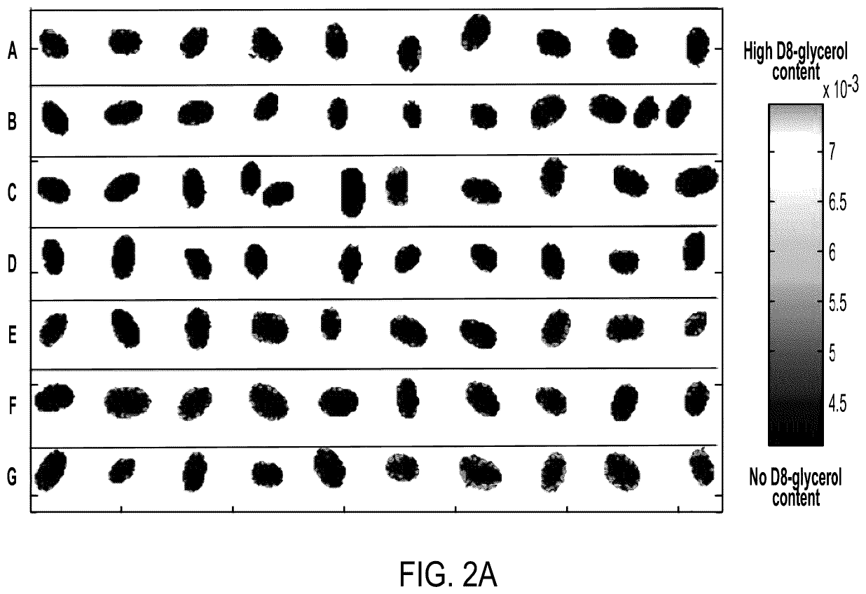

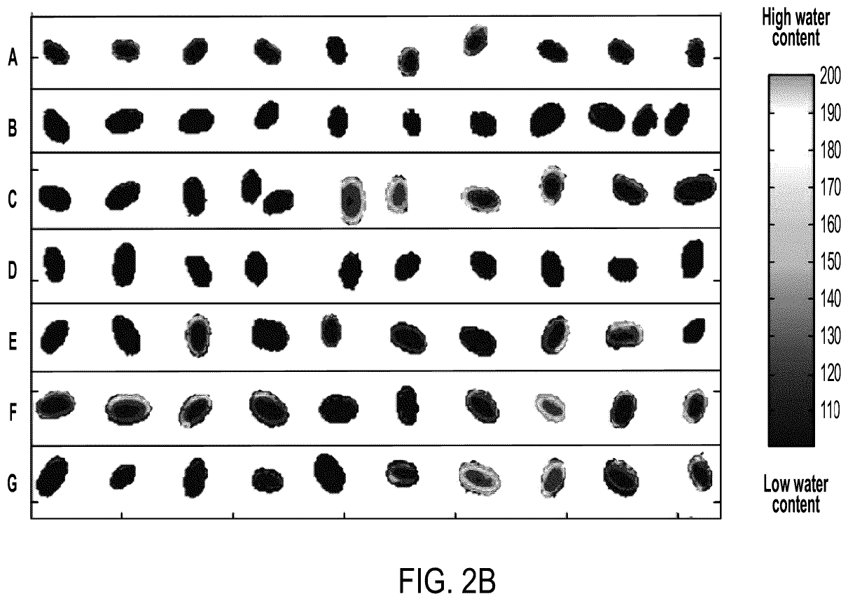

In order to evaluate water content and glycerol penetration into the hair fibers, Attenuated Total Reflection Fourier Transform Infrared (ATR-FTIR) microscopy was used, focusing on the 2100 cm 1 and 2200 cm 1 (C-D) region. FIGS. 2A (deuterated glycerol) and 2B (water) are images of cross sections of hair fibers from swatches A-G. ATR-FTIR images were recorded with a Lumos II system from Bruker with an ATR imaging accessory.

FIG. 2A shows that no deuterated glycerol was detected in the control swatch (A). Although some amount of deuterated glycerol has penetrated the hair of swatches B-C and E-F, the amount of penetration seen in swatch D is greater, and the amount in swatch G is significantly greater still. FIG. 2B shows the water content of the hair swatches, which correlates to the increased glycerol penetration.

Examples 4A-4B thus further confirm that treating hair with compositions according to the disclosure is surprisingly more effective at providing moisture benefits to hair.

The above examples confirm that compositions and methods according to the disclosure surprisingly and unexpectedly provide improved moisture, conditioning, and cosmetic benefits to keratinous substrates compared to compositions and methods not according to the disclosure.

1. A composition for treating keratinous substrates, comprising:
(a) at least one alkaline carbonate;
(b) at least one polyol;
(c) water and/or at least one non-aqueous solvent; and
(d) optionally at least one pH adjuster,
wherein the at least one carbonate and the at least one polyol are present in the composition in the form of an associated network.

2. The composition of claim 1, wherein the molar ratio of alkaline carbonate(s) to polyol(s) in the network ranges from less than 1:3 to about 1:50.

3. The composition of claim 1, wherein the at least one alkaline carbonate is chosen from sodium carbonate, calcium carbonate, potassium carbonate, magnesium carbonate, lithium carbonate, zinc carbonate, sodium bicarbonate, potassium bicarbonate, or combinations of two or more thereof.

4. The composition of claim 1, wherein the total amount of alkaline carbonates in the composition and/or associated network ranges from about 0.1% to about 10% by weight, relative to the total weight of the composition.

5. The composition of claim 1, wherein the at least one polyol is chosen from polyols with a molar mass of less than about 500 g/mol.

6. The composition of claim 1, comprising at least one polyol chosen from glycerol, sorbitol, xylitol, maltitol, isomalt, or a combination of two or more thereof.

7. The composition of claim 1, wherein the total amount of polyols in the composition and/or associated network ranges from about 1% to about 25% by weight, relative to the total weight of the composition.

8. The composition of claim 1, comprising:
(a) at least one alkaline carbonate;
(b) glycerol and optionally at least one additional polyol;
(c) water; and
(d) at least one pH adjuster,
wherein the molar ratio of alkaline carbonate(s) to polyol(s) in the network ranges from about 1:5 to about 1:15.

9. A method of treating keratinous substrates, the method comprising applying to the keratinous substrates a composition comprising:
(a) at least one alkaline carbonate;
(b) at least one polyol;
(c) water and/or at least one non-aqueous solvent; and
(d) optionally at least one pH adjuster,
wherein the at least one carbonate and the at least one polyol are present in the composition in the form of an associated network.

10. The method according to claim 9, wherein the molar ratio of alkaline carbonate(s) to polyol(s) in the network ranges from about 1:5 to about 1:15.

11. The method of claim 9, further comprising removing the composition from the keratinous substrates after a leave-on period ranging from about 1 minute to about 24 hours.

12. A simplex solution comprising:
(a) at least one alkaline carbonate;
(b) at least one polyol; and
(c) optionally water,
wherein the at least one carbonate and the at least one polyol are present in the simplex solution in the form of an associated network.

13. The simplex solution of claim 12, wherein the molar ratio of alkaline carbonate(s) to polyol(s) in the simplex solution ranges from less than 1:3 to about 1:50.

14. The simplex solution of claim 12, wherein the at least one alkaline carbonate is chosen from sodium carbonate, calcium carbonate, potassium carbonate, magnesium carbonate, lithium carbonate, zinc carbonate, sodium bicarbonate, potassium bicarbonate, or combinations of two or more thereof.

15. The simplex solution of claim 12, wherein the at least one polyol is chosen from polyols with a molar mass of less than about 500 g/mol.

16. The simplex solution of claim 12 comprising:
(a) at least one alkaline carbonate;
(b) glycerol and optionally at least one additional polyol; and
(c) optionally water,
wherein the molar ratio of alkaline carbonate(s) to polyol(s) ranges from about 1:5 to about 1:15.

17. A process for preparing a composition for treating keratinous substrates, the process comprising:
(i) preparing an associated network of alkaline carbonate(s) and polyol(s) by combining at least one alkaline carbonate, at least one polyol, and optionally water in a molar ratio of alkaline carbonate(s):polyol(s) that ranges from less than 1:3 to about 1:50 to form a simplex solution;
(ii) optionally heating the simplex solution;
(iii) adding at least one solvent chosen from water and/or non-aqueous solvents to the simplex solution to form the composition; and
(iv) optionally adding at least one pH adjuster to the composition.

18. The process of claim 17, comprising (ii) heating the simplex solution to a temperature ranging from about 35° C. to about 100° C. and subsequently cooling the simplex solution.

19. A composition prepared according to the process of claim 17.

20. A method of treating keratinous substrates, the method comprising applying the composition of claim 19 to the keratinous substrates.
